(19) United States
(12) Patent Application Publication
(10) Pub. No.: US 2025/0261967 A1
(43) Pub. Date: Aug. 21, 2025

(54) CONTROLLED BONE ACCESS AND OPERATOR FEEDBACK FEATURES

(71) Applicant: Relievant Medsystems, Inc., Edina, MN (US)

(72) Inventors: Neil Shigeo SASAKI, Edina, MN (US); Brian W. DONOVAN, Edina, MN (US); Benjamin David PARKER, Edina, MN (US); Alexander PRUITT, Edina, MN (US); Laura LUONG, Edina, MN (US)

(21) Appl. No.: 18/856,570

(22) PCT Filed: Apr. 7, 2023
(86) PCT No.: PCT/US2023/017913

§ 371 Date: Oct. 11, 2024

Related U.S. Application Data
(60) Provisional application No. 63/329,814, filed on Apr. 11, 2022.

(51) Int. Cl. A61B 17/34; A61B 90/00

(52) U.S. Cl. CPC A61B 17/3472; A61B 17/3421; A61B 90/03; A61B 2090/034

(57) ABSTRACT

Described herein are various implementations of systems and methods for controllably accessing and modulating tissue (for example, systems and methods for accessing and ablating nerves or other tissue within or surrounding a vertebral body to treat chronic low back pain).

### RELATED APPLICATIONS

This application claims priority to U.S. Provisional Application No. 63/329,814 filed Apr. 11, 2022, the entire content of which is hereby incorporated herein by reference.

### FIELD

Described herein are various implementations of systems, devices and methods to facilitate controlled access (for example, systems and methods for accessing locations within or surrounding a vertebral body or other bone) for performing diagnostic and/or therapeutic procedures (for example, intraosseous nerve ablation) within bone (e.g., vertebral body) or a body lumen or cavity or tissue. The systems, devices and methods may incorporate tactile and/or audible operator feedback to provide additional confidence and ease of use.

### BACKGROUND

Back pain is a very common health problem worldwide and is a major cause for work-related disability benefits and compensation. At any given time, low back pain impacts nearly 30% of the US population, leading to 62 million annual visits to hospitals, emergency departments, outpatient clinics, and physician offices. Back pain may arise from strained muscles, ligaments, or tendons in the back and/or structural problems with bones or spinal discs. The back pain may be acute or chronic. Existing treatments for chronic back pain vary widely and include physical therapy and exercise, chiropractic treatments, injections, rest, pharmacological therapy such as opioids, pain relievers or anti-inflammatory medications, and surgical intervention such as vertebral fusion, discectomy (e.g., total disc replacement), or disc repair. Existing treatments can be costly, addictive, temporary, ineffective, and/or can increase the pain or require long recovery times. In addition, existing treatments do not provide adequate relief for the majority of patients and only a small percentage are surgically eligible.

### SUMMARY

Applicant's existing technology (the Intracept® procedure by Relievant®) offers a safe and effective minimally invasive procedure that targets the basivertebral nerve and/or other intraosseous nerves (e.g., branches of a sinuvertebral nerve innervating one or both endplates of one or more vertebral bodies) residing in a vertebral body or within other bones for the relief of chronic vertebrogenic low back pain or other back pain or pain associated with other bones or joints. As disclosed herein, several embodiments of bone access tools incorporate features to facilitate controlled introduction and removal of various components of the bone access tools while also facilitating operator feedback and ease of use.

Other bones may also be accessed, such as bones associated with the legs, arms, knees, hips, feet, wrists, and hands. The bone access tools may facilitate curved access to intraosseous locations, or locations within bones. Straight bone access may alternatively be implemented.

A system or kit of the bone access tools may optionally include one or more access tools (e.g., stylets, cannulas, curettes, bone drills) configured to access a target nerve to be treated (e.g., basivertebral nerve and/or other branch of a sinuvertebral nerve and/or other intraosseous nerve). The kit may also or alternatively optionally include one or more treatment tools configured to modulate (e.g., ablate, stimulate, denervate, inhibit, necrose, electroporate, molecularly dissociate) the target nerve. The optional treatment tool includes one or a combination of the following: a radiofrequency (RF) energy delivery device, a microwave energy delivery device, an ultrasound energy delivery device, a cryomodulation device (e.g., cryoablation device), a laser energy delivery device, a vapor or steam delivery device, and/or a drug eluting device (e.g., chemical or fluid ablation device configured to elute a fluid capable of denervating or ablating a nerve, such as alcohol or phenol).

In accordance with several embodiments, easy, or simplified, tool exchange without loss of position during use of the bone access tools is desirable to facilitate ease of use, enhanced safety and efficacy, and repeatability. At several points throughout a bone access procedure (e.g., transpedicular, peri-pedicular, or extrapedicular access to a location within a vertebral body), introduction and removal of tools within and from other tools may cause inadvertent axial or translational or rotational movement of the tool or instrument within which other tools are being inserted or removed (e.g., an introducer cannula or curved cannula), which could result in incorrect placement if unnoticed. To help prevent the accidental movement and maintain part position and/or orientation during tool exchange, one or more detents or other mating features may be included on certain tools that interact with each other. In accordance with several embodiments, the addition of the one or more detents or other mating features may provide many benefits. For example, the addition of a detent or other mating feature may allow the operator to have a tactile (and potentially audible) feedback identifying when one tool component comes into contact (or sufficiently close contact) with a face or surface (e.g., an upper face or surface) of another tool component. As another example, the addition of one or more detents or other mating features may add significant friction to the assembly to prevent accidental movement when exchanging parts or tools. The detents or other mating features may act as an anti-rotation and/or -backdrive feature. As yet another example, the addition of a detent or other mating feature also may provide the operator more discrete control of tools during insertion or retraction. Without the detent or other mating feature, the operator may simply have to visually track the translation of tools with respect to each other. If a tactile or audible click is provided to the operator, instructions (oral or written) can be provided based on a number of clicks instead of, or in addition to, instructions requiring visual observation of one or more tool components by the operator. Embodiments described herein may provide increased operator confidence and verification.

In accordance with several configurations, a bone access system adapted to facilitate percutaneous access to a target treatment location within bone includes a first instrument (e.g., an introducer cannula) having a proximal handle and a distal elongate tube extending from the proximal handle (e.g., along a longitudinal axis). The proximal handle of the first instrument (e.g., introducer cannula) includes an opening (e.g., central opening, recess, or cavity) in its upper surface (e.g., adapted to receive a portion of another tool or instrument sized and adapted to be at least partially inserted within or through the first instrument (e.g., introducer cannula)). The distal elongate tube of the first instrument (e.g., introducer cannula) may include a lumen accessible via the opening (e.g., central opening) of the proximal handle of the first instrument (e.g., introducer cannula). The system also includes a second instrument (e.g., an access instrument, diagnostic instrument, treatment instrument) sized and configured to be inserted through the central opening and along the lumen of the first instrument (e.g., introducer cannula) until a distal end portion of the second instrument (e.g., access instrument) extends beyond an open distal tip of the first instrument (e.g., introducer cannula). The first instrument (e.g., introducer cannula) and the second instrument (e.g., access instrument) together include a tactile feedback mechanism adapted to provide tactile feedback indicative of interaction between the second instrument (e.g., access instrument) and the first instrument (e.g., introducer cannula).

In some configurations, the tactile feedback mechanism includes corresponding mating features (e.g., detent features) of the introducer cannula and the access instrument designed to interact with each other upon alignment of the respective corresponding mating features (e.g., detent features). The corresponding mating features may be positioned at various locations or surfaces of the introducer cannula and the access instrument, or other tools or instruments that are configured to interface with each other (e.g., other cannulas, guide systems, endoscopic devices, diagnostic devices, treatment devices, etc.).

In some configurations, the tactile feedback mechanism includes a tooth or protrusion on one side and a plurality of indentations on the other side distributed to form a pattern (e.g., circle), wherein the tooth or protrusion is adapted to engage with one of the plurality of indentations. For example, the tooth or protrusion can be formed on the introducer cannula and the plurality of indentations can be formed on the access instrument, or vice versa.

In some configurations, the tactile feedback mechanism includes corresponding magnets of the introducer cannula and the access instrument designed to interact with each other upon alignment of the respective corresponding magnets.

In some configurations, the tactile feedback mechanism includes hook-and-loop fasteners.

In some configurations, the tactile feedback mechanism includes a wave washer adapted to provide an interference fit between the access instrument and the introducer cannula.

In some configurations, the tactile feedback mechanism includes a spring detent within the central opening of the introducer cannula that is configured to interact with corresponding detent features of the access instrument.

In several configurations, the tactile feedback mechanism produces audible clicks.

In some configurations, the tactile feedback mechanism causes a change in an amount of torque required to continue to advance the access instrument distally within the introducer cannula.

The access instrument may include a cannula having a pre-curved distal end portion. The access instrument may include a straight cannula or a treatment probe or diagnostic instrument. The access instrument may include a proximal handle adapted to be malleted or pressed by an operator to facilitate distal advancement of the access instrument. The introducer cannula and the access instrument may be sized to facilitate access to an intraosseous location within a vertebral body through a percutaneous incision.

In accordance with several configurations, a bone access system adapted to facilitate percutaneous access to a target treatment location within bone (e.g., within a vertebral body) includes an introducer cannula having a proximal handle and a distal elongate tube extending from the proximal handle. The proximal handle of the introducer cannula includes a central opening in its upper surface. The upper surface (e.g., central opening) of the proximal handle of the introducer cannula includes one or more detents (e.g., first detent(s)) or mating features projecting radially inward from an inner surface of the central opening. The distal elongate tube of the introducer cannula includes a lumen accessible via the central opening of the proximal handle of the introducer cannula. The bone access system also includes an access instrument sized and configured to be inserted through the central opening and along the lumen of the introducer cannula until a distal end portion of the access instrument extends beyond an open distal tip of the introducer cannula. The access instrument includes an elongate shaft having a threaded proximal portion and a gear (e.g., gear wheel) mechanically coupled to the threaded proximal portion and adapted to be translated proximally and distally along the threaded proximal portion via rotation of the gear wheel. The access instrument may optionally include a generally smooth (e.g., non-threaded) distal portion. The gear wheel includes an annular flange that includes a second detent feature which is a plurality of detent elements (e.g., indentations) spaced around a circumference (e.g., cylindrical lateral surface) of a lower surface of the annular flange. The second detent feature is adapted to mechanically engage with the one or more first detents on the proximal handle (e.g., within the central opening) of the introducer cannula so as to provide tactile and/or audible feedback to an operator upon engagement of the plurality of detent elements of the access instrument with the one or more first detents (e.g., teeth) of the introducer cannula so as to provide controlled advancement of the access instrument with respect to the introducer cannula.

The second detent feature of access instrument (e.g., of the annular flange) may include a plurality of indentations, notches, recesses, indentations, divots, or other mating features. Each of the plurality of notches, recesses, indentations, divots or other mating features of the second detent feature may comprise a semi-circular shape, a sawtooth shape, a triangular shape, a square shape, a sinusoidal shape, or other shape. The first detent feature (e.g., one or more detents) of the introducer cannula may include teeth, protrusions, tabs, bosses, nubs, or other mating features formed on or along the upper and/or inner surface of the introducer cannula (e.g., about the central opening). In some embodiments, the second detent feature of the access instrument may include teeth, protrusions, tabs, bosses, nubs, or other mating features, and the first detent feature of introducer cannula may include a plurality of indentations, notches, recesses, indentations, divots, or other mating features as long as the first detent feature and the second detent feature can be tactilely engaged.

In some configurations, the plurality of detent (e.g., notch or indentation) elements of the second detent feature can be uniformly spaced around a circumference of the annular flange of the gear wheel of the access instrument. In other configurations, the plurality of detent elements of the second detent feature are not uniformly spaced.

The one or more detents (e.g., teeth) and the plurality of detent (e.g., indentation) elements may produce an audible click upon engagement and/or disengagement. Engagement between the one or more detents (e.g., teeth) of the introducer cannula and a respective one of the plurality of detent (e.g., indentation) elements of the annular flange may result in an increase in an amount of torque required to disengage the one or more detents (e.g., teeth) from the respective one of the plurality of detent (e.g., indentation) elements. In accordance with several embodiments, the increase in required torque may advantageously help to prevent inadvertent rotational and/or translational movement of one instrument or tool with respect to the other (e.g., as additional tools are inserted or withdrawn from the access instrument and/or introducer cannula).

In some systems, the access instrument is a cannula having a pre-curved distal end portion. In some systems, the access instrument includes a proximal handle adapted to be malleted or pressed by an operator to facilitate distal advancement of the access instrument.

In some configurations the first detent feature (e.g., one or more detents) of the introducer cannula consists of a single detent (e.g., tooth) and not multiple detents (e.g., teeth).

In some configurations, the central opening of the introducer cannula may include an abutment member extending radially inward from the inner surface of the central opening that is adapted to abut against a lower surface of the annular flange of the gear wheel of the access instrument upon advancement of the access instrument within the introducer cannula.

In some implementations, the introducer cannula and the access instrument are sized to facilitate access to an intraosseous location within a vertebral body through a percutaneous incision.

In some implementations, the proximal handle of the introducer cannula may include a curved or ramped insertion slot.

In accordance with several embodiments, a bone access system includes an introducer cannula having an introducer handle at a proximal end and a distal elongated tube attached to and extending from the introducer handle along a longitudinal axis. The introducer handle includes an abutment surface disposed about an upper surface or within an opening, cavity, or recess within the upper surface. The abutment surface includes a first mating feature (e.g., detent feature) formed thereon or therein. A lumen extends through the introducer cannula from the introducer handle to an open distal tip of the distal elongated tube along the longitudinal axis. The system also includes an access instrument having an instrument handle at a proximal end and a distal shaft portion attached to and extending from the instrument handle along the longitudinal axis. The distal shaft portion of the access instrument is configured to be received into the lumen of the introducer cannula. The instrument handle includes an abutment surface (e.g., a lower surface and/or side surface) having a second mating feature (e.g., detent feature) formed thereon or therein. The second mating feature is configured to mate with the first mating feature of the introducer cannula. When the distal shaft portion of access instrument is inserted into the lumen of the introducer cannula from the proximal end and the abutment surface (e.g., lower surface and/or side surface) of the instrument handle is close to or in contact or touch with the abutment surface of the introducer handle, the first mating feature (e.g., detent feature) of the abutment surface is mated with the second mating feature (e.g., detent feature) of the abutment surface of the instrument handle.

The mating of the features may comprise a removable mating engagement that can be overcome with sufficient force (e.g., torque or pressure). For implementations involving detent features, when the first detent feature on the abutment surface is mated with the second detent feature on the lower surface of the instrument handle, rotation of the second detent feature around the longitudinal axis against the first detent feature causes a tactile response. When the first detent feature on the abutment surface of the introducer handle is mated with the second detent feature on the corresponding abutment surface of the instrument handle, rotation of the second detent feature around the longitudinal axis against the first detent feature may result in an audible clicking response and/or a tactile response that provides tactile or haptic feedback to the operator.

In some implementations, when the first detent feature on the abutment surface is mated with the second detent feature on the lower surface of the instrument handle, rotation of the second detent feature around the longitudinal axis against the first detent feature needs to overcome a torque of a predetermined amount in order to cause the detent features to disengage from each other, thereby providing a safety mechanism against inadvertent or undesired movement of the tools or instruments with respect to each other until movement is desired.

In some implementations, the access instrument further includes an extension portion extended distally from the instrument handle, the extension portion having an external thread formed thereon. The access instrument may also include a wheel including a hole along a central axis of the wheel, with the hole including an internal thread formed therein. The internal thread of the wheel may be configured to engage with the external thread of the extension portion. The wheel may be coupled with the extension portion with the internal thread of the wheel engaged with the external thread of the extension portion such that rotating the wheel causes the wheel to translate distally or proximally along the access instrument.

In some implementations, the first detent feature on the abutment surface of the introducer handle is one or more teeth and the second detent feature on the abutment surface of the instrument handle is a plurality of indentations, wherein when the first detent feature is mated with the second detent feature each of the one or more teeth bites into one of the plurality of the indentations.

In some implementations, the first detent feature on the abutment surface of the introducer handle is a plurality of indentations and the second detent feature on the abutment surface of the instrument handle is one or more teeth, wherein when the first detent feature is mated with the second detent feature each of the one or more teeth bites into one of the plurality of the indentations.

The plurality of indentations may form a circle around the longitudinal axis of the introducer cannula or the access instrument. The plurality of indentations of the circle may be equally or uniformly spaced or otherwise spaced at appropriate intervals.

In some implementations, the plurality of indentations are formed on a cylindrical lateral circumferential surface coaxial with the longitudinal axis.

The one or more teeth may be a single tooth, two teeth, three teeth, four teeth, or more than four teeth.

In various configurations, the plurality of indentations may be a semi-circular shape, a sawtooth shape, a triangular shape, a square shape, or a sinusoidal shape.

In accordance with several embodiments, the introducer cannula and the access instrument may be sized to facilitate access to an intraosseous location within a vertebral body through a percutaneous incision.

In some examples, an introducer cannula for penetrating into a bone includes an introducer handle at a proximal end, the introducer handle including an abutment surface disposed about an upper surface or within a recess or cavity of the upper surface, the abutment surface having a first mating (e.g., detent) feature formed thereon or therein. The introducer cannula also includes a distal elongated tube attached to and extending from the introducer handle along a longitudinal axis. A lumen extends through the introducer cannula from the introducer handle to an open distal tip of the distal elongated tube along the longitudinal axis. The first mating (e.g., detent) feature is configured to mate with a second corresponding detent feature formed on an access instrument adapted to be at least partially inserted within the introducer cannula.

In some implementations, when the first detent feature on the abutment surface is configured to mate with the second detent feature on the access instrument, rotation of the second detent feature around the longitudinal axis against the first detent feature causes a tactile response. In some implementations, when the first detent feature on the abutment surface is configured to mate with the second detent feature on the access instrument, rotation of the second detent feature around the longitudinal axis against the first detent feature results in an audible clicking response.

In some implementations, the first detent feature on the abutment surface of the introducer handle is one or more teeth and the second detent feature on the access instrument is a plurality of indentations, wherein when the first detent feature is mated with the second detent feature each of the one or more teeth bites into one of the plurality of the indentations.

In other implementations, the first detent feature on the abutment surface of the introducer handle is a plurality of indentations and the second detent feature on the access instrument is one or more teeth, wherein when the first detent feature is mated with the second detent feature each of the one or more teeth bites into one of the plurality of the indentations.

In some examples, a bone access instrument for penetrating into a bone includes an instrument handle at a proximal end and a distal shaft portion extending from the instrument handle along a longitudinal axis. The instrument handle includes an abutment surface (e.g., a lower and/or side surface) having a respective mating feature (e.g., a second detent feature) formed thereon or therein, the mating feature configured to mate with a corresponding mating feature (e.g., first detent feature) of an introducer cannula. The distal shaft portion is configured to be received into a lumen formed in the introducer cannula.

In some implementations, when the second detent feature on the lower surface of the instrument handle is configured to mate with the first detent feature of an introducer cannula, rotation of the second detent feature around the longitudinal axis against the first detent feature causes a tactile response.

In some implementations, when the second detent feature on the abutment surface of the instrument handle is configured to mate with the first detent feature of an introducer cannula, rotation of the second detent feature around the longitudinal axis against the first detent feature results in an audible clicking response.

In some implementations, when the second detent feature on the abutment surface of the instrument handle is configured to mate with the first detent feature of an introducer cannula, rotation of the second detent feature around the longitudinal axis against the first detent feature needs to overcome a torque of a predetermined amount.

In some implementations, the bone access instrument further includes an extension portion extending distally from the instrument handle, wherein the extension portion has an external thread formed thereon, The access instrument includes a wheel having a hole along a central axis of the wheel, with the hole having an internal thread formed therein configured to engage with the external thread of the extension portion. The wheel is coupled with the extension portion with the internal thread of the wheel engaged with the external thread of the extension portion, such that rotating the wheel causes the wheel to translate distally or proximally along the extension portion.

In some implementations, the second detent feature on the lower surface of the instrument handle is one or more teeth and the first detent feature on the abutment surface of the introducer handle is a plurality of indentations, wherein when the second detent feature is mated with the first detent feature each of the one or more teeth bites into one of the plurality of the indentations.

In some implementations, the second detent feature on the lower surface of the instrument handle is a plurality of indentations and the first detent feature on the abutment surface of the introducer handle is one or more teeth, wherein when the second detent feature is mated with the first detent feature each of the one or more teeth bites into one of the plurality of the indentations.

In some implementations, a method for facilitating percutaneous access to a target treatment location within a bone (e.g., vertebral body of a lumbar vertebra, sacral vertebra, thoracic vertebra, or cervical vertebra) includes inserting a distal portion of an introducer assembly to at least an outer cortical region of the bone. The introducer assembly includes an introducer cannula and an introducer stylet. The introducer cannula includes a proximal introducer handle at a proximal end and a distal elongated tube attached to and extending from the introducer handle, the elongated tube defining a longitudinal axis. The introducer cannula has a lumen formed therethrough from the proximal introducer handle to a distal tip of the elongated tube. The introducer stylet is configured to be received into the lumen of the introducer cannula. The method further includes removing the introducer stylet from the lumen of the introducer cannula and then inserting a bone access instrument or assembly (e.g., a curved cannula assembly) into the lumen. The curved cannula assembly may include a curved cannula handle and a shaft portion extended from the curved cannula handle, the shaft portion configured to be received into the lumen, the shaft portion having a distal curved portion. The method also includes advancing at least a distal portion of the bone access instrument (e.g., the distal curved portion of the curved cannula assembly) out of the distal tip of the introducer cannula toward the target treatment location within the bone (e.g., an ablation location where a lesion or heating zone may be formed sufficient to ablate one or more nerves within the bone). The introducer handle includes a first detent feature formed thereon and the bone access instrument (e.g., a component of the curved cannula handle, such as a flange of a gearwheel) includes a second detent feature formed thereon configured to mate with the first detent feature so as to provide tactile and/or audible feedback to an operator upon engagement of the second detent feature and the first detent feature so as to provide controlled advancement of the bone access instrument (e.g., curved cannula assembly) with respect to the introducer cannula.

In some implementations, the mating between the first detent feature of the introducer handle and second detent feature of the curved cannula handle allows a radial positioning of the curved cannula assembly within the introducer cannula.

In some implementations, the bone access instrument (e.g., curved cannula assembly) includes a gearwheel or other translation mechanism coupled to a proximal portion of the bone access instrument (e.g, the curved cannula handle). A lower flange or other member extending from the gearwheel, or a lower surface of the gearwheel itself, may include the second detent feature. Rotation of the gearwheel or other translation mechanism may adjust an axial position of the gearwheel on the proximal portion of the bone access instrument (e.g., curved cannula handle). Engagement of the gearwheel with the introducer handle may define the longitudinal position of the bone access instrument (e.g., curved cannula assembly) within the introducer cannula.

In some implementations, continued rotation of the gearwheel or other translation mechanism provides further tactile and/or audible feedback via the interaction of the first detent feature with the second detent feature.

The first detent feature and/or the second detent feature may comprise a plurality of detent elements. The first detent feature may comprise a single element and the second detent feature may comprise a plurality of detent elements, or vice-versa. The respective detent elements may comprise female elements (e.g., notches, grooves, recesses, etc.) or male elements (e.g., nubs, protrusions, teeth, etc.) such that the detent elements of the respective components are configured to removably engage with each other when sufficient torque is provided to disengage.

In some implementations, advancing the distal portion of the bone access instrument (e.g., the curved distal portion of the curved cannula assembly) out of the distal tip of the introducer cannula comprises malleting the proximal handle of the bone access instrument (e.g., the curved cannula handle). The method may also include advancing a treatment instrument through the access instrument and performing a treatment procedure within the bone. The treatment procedure may include applying thermal energy at the target treatment location sufficient to modulate (e.g., ablate) one or more nerves. The method may include advancing a diagnostic instrument through the access instrument and performing a diagnostic procedure within the bone. The bone may be a vertebral body and the treatment procedure may be a treatment procedure to treat chronic low back pain or other back pain.

Again, in some implementations, the methods described herein may be used in connection with tools that are to be inserted within tissue other than bone or within body lumens, cavities, recesses, or spaces (e.g, endoscopic, laparoscopic, intravascular, or intraluminal procedures).

In accordance with several embodiments, the systems and methods described herein advantageously do not rely on visual observation alone to access a treatment and/or diagnostic location. Tactile and/or audible feedback is enabled to facilitate controlled access.

The methods described herein may also include one or more diagnostic and/or treatment steps after controllably accessing the diagnostic and/or treatment location. In some implementations, treatment procedures after access to the bone may include modulation of nerves within or surrounding bones. The terms “modulation” or “neuromodulation”, as used herein, shall be given their ordinary meaning and shall also include ablation, permanent denervation, temporary denervation, disruption, blocking, inhibition, electroporation, therapeutic stimulation, diagnostic stimulation, inhibition, necrosis, desensitization, or other effect on tissue. Neuromodulation shall refer to modulation of a nerve (structurally and/or functionally) and/or neurotransmission. Modulation is not necessarily limited to nerves and may include effects on other tissue, such as tumors or other soft tissue.

Several embodiments of the disclosed technology have one or more of the following advantages: (i) tactile feedback identifying interaction between components of a bone access system or kit; (ii) audible feedback identifying interaction between components of a bone access system or kit; (iii) prevention of inadvertent movement or translation between components of a bone access system or kit; (iv) discrete control of relative movement between components of a bone access system or kit; and/or (v) ease of use in accessing bone utilizing a bone access system or kit.

For purposes of summarizing the disclosure, certain aspects, advantages, and novel features of embodiments of the disclosure have been described herein. It is to be understood that not necessarily all such advantages may be achieved in accordance with any particular embodiment of the disclosure provided herein. Thus, the embodiments disclosed herein may be embodied or carried out in a manner that achieves or optimizes one advantage or group of advantages as taught or suggested herein without necessarily achieving other advantages as may be taught or suggested herein.

The methods summarized above and set forth in further detail below describe certain actions taken by a practitioner; however, it should be understood that they can also include the instruction of those actions by another party. Thus, actions such as “applying thermal energy” include “instructing the applying of thermal energy.” Further aspects of embodiments of the disclosure will be discussed in the following portions of the specification. With respect to the drawings, elements from one figure may be combined with elements from the other figures.

### BRIEF DESCRIPTION OF THE DRAWINGS

Several embodiments of the disclosure will be more fully understood by reference to the following drawings which are for illustrative purposes only:

FIG. 1 illustrates an example kit or system of access tools configured to access a vertebral body or other bone.

FIG. 2A is a side view of an introducer cannula of the kit or system of FIG. 1. FIG. 2B is a top view of the introducer cannula.

FIG. 3A is a side view of a curved cannula of the kit or system of FIG. 1. FIG. 3B is a close-up side view of a proximal end portion of the curved cannula.

FIGS. 3C and 3D illustrate operation of a gear wheel of the curved cannula to facilitate prevention or advancement of the curved cannula assembly within the introducer cannula at different stages of a bone access method utilizing the kit or system of FIG. 1.

FIG. 3E illustrates a side view of the curved cannula inserted within the introducer cannula, with the introducer cannula being transparent.

FIG. 4A shows a close-up side view of an example of a gear wheel of the curved cannula illustrating detent elements of the gear wheel.

FIG. 4B shows a perspective top view of a portion of a proximal handle of the introducer cannula illustrating an example of a detent element of the introducer cannula.

FIG. 4C illustrates a side view of a curved cannula including the gear wheel shown in FIG. 4A inserted within the proximal handle of the introducer cannula shown in FIG. 4B, with the introducer cannula being transparent.

FIG. 4D illustrates a side cross-section view of a proximal portion of the curved cannula.

FIGURES SA, 5B, 5C, and 5D illustrate additional examples of detent elements of the gear wheel of the curved cannula.

FIG. 6 illustrates an example of a curved cannula and introducer cannula that include magnets to facilitate controlled introduction and removal of the curved cannula within the introducer cannula.

FIG. 7 illustrates an example of a curved cannula and introducer cannula that incorporate the use of a spring detent mechanism.

FIGS. 8 and 9 illustrate examples of interference designs that incorporate the use of an interfacing material or interference fit approach to change tactile feedback as the gear wheel of the curved cannula comes into contact with the introducer cannula.

FIGS. 10A-10E illustrate various steps of a method of accessing and treating tissue within a vertebral body using one or more of the access tools of the kit or system of FIG. 1.

### DETAILED DESCRIPTION

Several implementations described herein are directed to systems, devices and methods for facilitating controlled access to locations within bones. In some implementations, an intraosseous nerve (e.g., basivertebral nerve and/or other intraosseous nerve branching from a sinuvertebral nerve) within a bone (e.g., vertebral body) of the spine is accessed for diagnostic or treatment purposes (e.g., treatment or prevention of chronic low back pain). The vertebral body may be located in any level of the vertebral column (e.g., cervical, thoracic, lumbar and/or sacral). Multiple vertebral bodies may be accessed simultaneously or sequentially. The multiple vertebral bodies may be located in a single spine segment (e.g., two adjacent vertebral bodies in the sacral spine segment (e.g., S1 and S2) or lumbar spine segment (e.g., L3, L4 and/or L5) or thoracic spine segment or cervical spine segment), or in different spine segments (e.g., an L5 vertebra in the lumbar spine segment and an SI vertebra in the sacral spine segment). Intraosseous nerves within bones other than vertebral bodies may also be modulated. For example, nerves within a humerus, radius, femur, tibia, calcaneus, tarsal bones, hips, knees, and/or phalanges may be accessed. In some embodiments, the shoulder is treated. In several embodiments, the devices and methods described herein are used for the diagnosis and/or treatment of back pain, nerve stimulation or ablation, intravascular or intraluminal applications, and endoscopic applications.

In some implementations, the one or more nerves being modulated are extraosseous nerves located outside the vertebral body or other bone (e.g., at locations before the nerves enter into, or after they exit from, a foramen of the bone). Other tissue in addition to, or alternative to, nerves may also be treated or otherwise affected (e.g., tumors or other cancerous tissue or fractured bones). Portions of nerves within or on one or more vertebral endplates or intervertebral discs between adjacent vertebral bodies may be modulated.

The modulation of nerves or other tissue may be performed to treat one or more indications, including but not limited to chronic low back pain, upper back pain, acute back pain, joint pain, tumors in the bone, and/or bone fractures. The pain may originate from one or multiple vertebrae and/or from one or more intravertebral discs. The modulation of nerves may also be performed in conjunction with bone fusion or arthrodesis procedures so as to provide synergistic effects or complete all-in-one, “one-and-done” treatment that will not require further surgical or minimally invasive interventions. In some embodiments, non-bone diagnostics and therapeutics are facilitated by the devices and methods described herein, such as intraluminal or tissue access (e.g., intravascular, endoscopic, intestinal, esophageal, etc.).

In accordance with several implementations, the systems and methods of treating back pain or facilitating neuromodulation of intraosseous nerves described herein can be performed without surgical resection, without general anesthesia, without cooling (e.g., without cooling fluid), and/or with virtually no blood loss. In some embodiments, the systems and methods of treating back pain or facilitating neuromodulation of intraosseous nerves described herein facilitate easy retreatment if necessary. In accordance with several implementations, successful treatment can be performed in challenging or difficult-to-access locations and access can be varied depending on bone structure or differing bone anatomy. One or more of these advantages also apply to treatment of tissue outside of the spine (e.g., other orthopedic applications or other tissue).

Access tools may include an introducer assembly including an outer cannula and a sharpened stylet, an inner cannula configured to be introduced through the outer cannula, and/or one or more additional stylets, curettes, or drills to facilitate access to an intraosseous location within a vertebral body or other bone. The access tools (e.g., outer cannula, inner cannula, stylets, curettes, drills) may have pre-curved distal end portions or may be actively steerable or curveable. Any of the access tools may have beveled or otherwise sharp tips or they may have blunt or rounded, atraumatic distal tips. Curved drills may be used to facilitate formation of curved access paths within bone. Straight access tools may also be used. Any of the access tools may be advanced over a guidewire in some implementations.

The access tools may be formed of a variety of flexible materials (e.g., ethylene vinyl acetate, polyethylene, polyethylene-based polyolefin elastomers, polyetheretherketone, polypropylene, polypropylene-based elastomers, styrene butadiene copolymers, thermoplastic polyester elastomers, thermoplastic polyurethane elastomers, thermoplastic vulcanizate polymers, metallic alloy materials such as nitinol, and/or the like). Combinations of two or more of these materials may also be used. The access tools may include chevron designs or patterns or slits along the distal end portions to increase flexibility or bendability. Any of the access tools may be manually or automatically rotated (e.g., using a robotic control system) to facilitate a desired trajectory.

In some implementations, an outer cannula assembly (e.g., introducer assembly) includes a straight outer cannula and a straight stylet configured to be received within the outer cannula. The outer cannula assembly may be inserted first to penetrate an outer cortical shell of a bone and provide a conduit for further access tools to the inner cancellous bone. An inner cannula assembly may include a cannula having a pre-curved or steerable distal end portion and a stylet having a corresponding pre-curved or steerable distal end portion. Multiple stylets having distal end portions with different curvatures may be provided in a kit and selected from by a clinician. The inner cannula assembly may alternatively be configured to remain straight and non-curved.

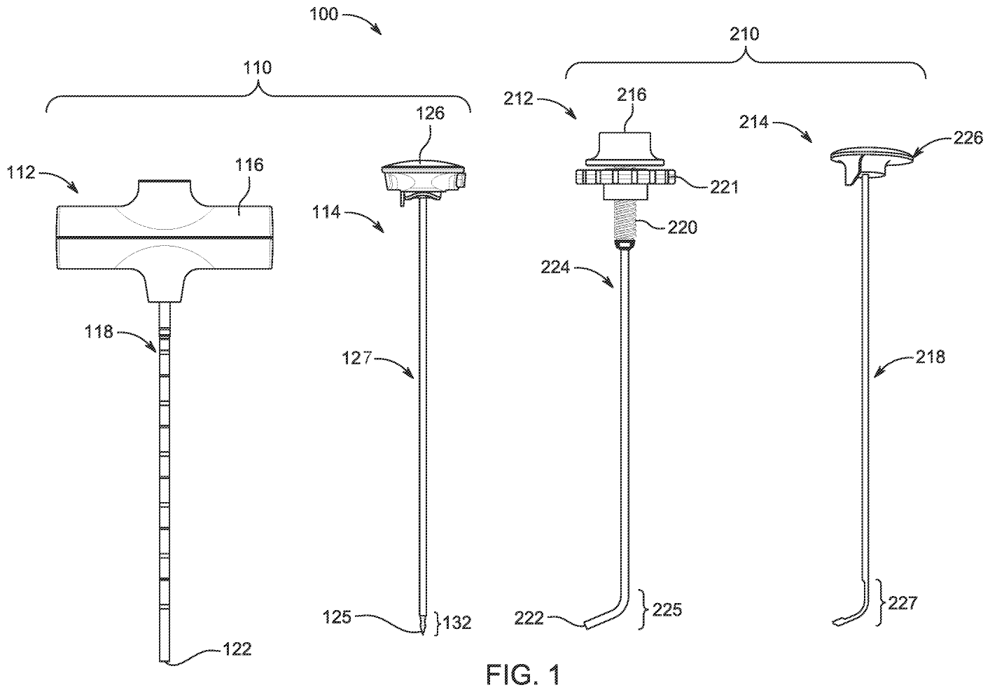

With reference to FIG. 1, in one implementation, a kit or system 100 of access tools (e.g., bone access tools) may include an introducer assembly 110 comprised of an introducer cannula 112 and an introducer stylet 114, and a curved cannula assembly 210 comprised of a curved cannula 212 and a corresponding curved stylet, or J-stylet 214.

The introducer cannula 112 may include a proximal introducer handle 116 and a distal elongate tube 118 (e.g., hypotube) extending from the introducer handle 116 along a longitudinal axis. The illustrated introducer handle 116 may comprise a “smokestack” or “T-Handle” design configuration adapted to provide sufficient finger clearance and gripping to facilitate advancement of removal of the introducer cannula 112. However, alternative design configurations for the proximal handle other than a “smokestack” or “T-handle” design may be incorporated.

The introducer stylet 114 is configured to be received in a lumen of the introducer cannula 112 in a manner such that a distal tip 125 of the introducer stylet 114 protrudes from an open distal tip 122 of the introducer cannula 112, thereby forming the introducer assembly 110 in combination. The introducer stylet 114 includes a proximal first handle 126 and a distal elongate member 127. The first handle 126 comprises an upper surface that is adapted for malleting by a mallet and a lower surface that is adapted to facilitate removal of the introducer stylet 114 by an operator. The length of the distal elongate member 127 may range from 8 mm to 14 mm (e.g., 8 mm, 8.5 mm, 9 mm, 9.5 mm, 10 mm, 10.5 mm, 11 mm, 11.5 mm, 12 mm, 12.5 mm, 13 mm, 13.5 mm, 14 mm). The introducer stylet 114 may be bevel tipped, trocar tipped, cone tipped, and/or diamond tipped. A distal end portion 132 of the introducer stylet 114 may optionally comprise a scalloped section to provide a release mechanism for bone compaction. The scalloped section may be designed to have a side profile shaped generally like an hourglass. The distal tip 125 of the distal end portion 132 may comprise a full diameter so as to be adapted to break apart bone (e.g., pedicle bone, cortical bone of a vertebral body). As the bone is broken up by the distal tip 125 of the distal end portion 132, bone shards or chips can pack into a gap formed between the distal end portion 132 of the introducer stylet 114 and the inner surface of the distal end portion of the introducer cannula 112, thereby making it more difficult for the introducer stylet 114 to be removed from the introducer cannula 112. In accordance with several embodiments, the scalloped section of the introducer stylet 114 may advantageously provide the bone shards and fragments a place to fall into during removal of the introducer stylet 114 so as to facilitate easier removal of the introducer stylet 114.

The curved cannula 212 includes a proximal second handle (e.g., instrument handle, curved cannula handle) 216, a wheel feature (e.g., a gear wheel) 221, and a threaded proximal extension portion 220 extended distally from the proximal second handle (e.g., instrument handle) 216. The gear wheel 221 is disposed about the extension portion 220 and translates upon rotation of the gear wheel 221. The curved stylet 214 comprises a proximal handle 226. The curved stylet 214 may be one of a plurality of access instruments that can be received in a lumen of the curved cannula 212 in a manner such that a distal tip of the curved stylet 214 protrudes from an open distal tip 222 of the curved cannula 212, thereby forming the curved cannula assembly 210 in combination. The curved cannula 212 and the curved stylet 214 may each comprise a straight proximal main body portion and a curved distal end portion 225, 227. The curves of the curved distal end portions 225, 227 of the curved cannula 212 and the curved stylet 214 may correspond to each other. The kit or system 100 may optionally include a straight stylet (not shown) that is a flexible channeling stylet configured to be delivered through the curved cannula 212 and then to form and maintain a straight or generally straight path upon exiting the open distal tip 222 of the curved cannula 212.

The access tools (e.g., bone access tools) may be provided as a kit that may optionally additionally include one or more additional introducer cannulas, one or more additional introducer stylets (e.g., with different tips, such as one with a bevel tip and one with a diamond or trocar tip), one or two or more than two additional curved cannulas (e.g., having a curved distal end portion of a different curvature than a first curved cannula), an additional curved stylet (e.g., having a different curvature or different design configured to access hard bone), an introducer drill, and/or an additional straight stylet (e.g., having a different length than the first straight stylet. Some kits may include optional additional access tool components or accessory kit modules adapted to access one or more additional vertebrae in the same spinal segment or in different spinal segments. The kit 100 may also include one or more (e.g., at least two) treatment devices (such as radiofrequency energy delivery probes).

In some embodiments, the access tools (e.g., kit 100) may be specifically designed and adapted to facilitate access to hard, non-osteoporotic bone (e.g., bone surrounding or within a vertebral body, such as a cervical vertebra, a thoracic vertebra, a lumbar vertebra, or a sacral vertebra). Hard bone may be determined based on bone mass density testing, compressive strength determinations, compressive modulus determinations, imaging modalities, or based on tactile feel by the operator as access instruments are being advanced. In some implementations, hard bone may be determined as bone having a bone mineral density score within a standard deviation of a normal healthy young adult (e.g., a T score greater than or equal to −1). In some implementations, hard bone may be identified as bone having a compressive strength of greater than 4 MPa and/or a compressive modulus of greater than 80 MPa for cancellous bone and greater than 5.5 MPa and/or a compressive modulus of greater than 170 MPa for cortical bone. Some kits may include at least two of every access instrument. Some kits may include optional add-on components or accessory kit modules for accessing hard bone (e.g., an introducer drill and a curved stylet 214 specially configured to access hard bone).

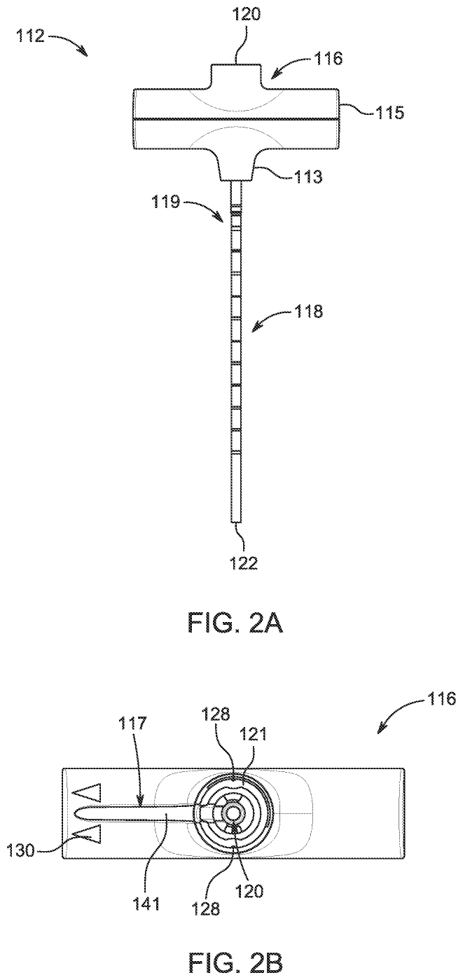

FIG. 2A is a side view of the introducer cannula 112 of the kit or system 100 of FIG. 1. FIG. 2B is a top view of the introducer cannula 112.

The introducer handle 116 includes a lower portion 113 (e.g., bulged portion or flange) extending downward from a lower surface of a crossbar portion 115. The introducer handle 116 includes an upper central opening 120 (e.g., recess, cavity) configured to facilitate straight axial insertion of the introducer stylet 114 or other access tools (e.g., straight access tools). The upper central opening 120 may be positioned so as to correspond with (e.g., be coaxial with) a central lumen extending through the elongate tube 118 of the introducer cannula 112 so as to facilitate insertion of access instruments (e.g., introducer stylet 114, steerable cannulas or steerable stylets) therethrough. The introducer handle 116 may also include coupling features 121 (e.g., recesses, notches, grooves, tabs) to facilitate coupling or mating of a second handle 216 of the introducer stylet 114 with the introducer handle 116 of the introducer cannula 112. The coupling features 121 may be adapted to prevent rotation of the introducer stylet 114 and/or to provide assurance that the distal tip 125 of the introducer stylet 114 extends beyond an open distal tip 122 of the tube 118 of the introducer cannula 112 so as to enable penetration of the distal tip 125 of the introducer stylet 114 through bone. The upper surface of the introducer handle 116 of the introducer cannula 112 may also include a curved lateral slot 117 and curved ramp 141 to facilitate insertion of the curved cannula assembly 210 into the introducer handle 116 and then into and along the central lumen of the tube 118.

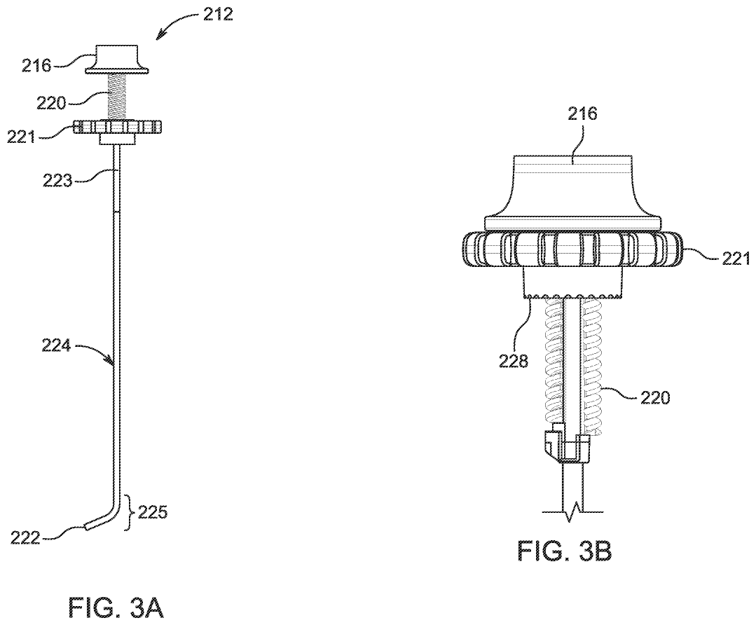

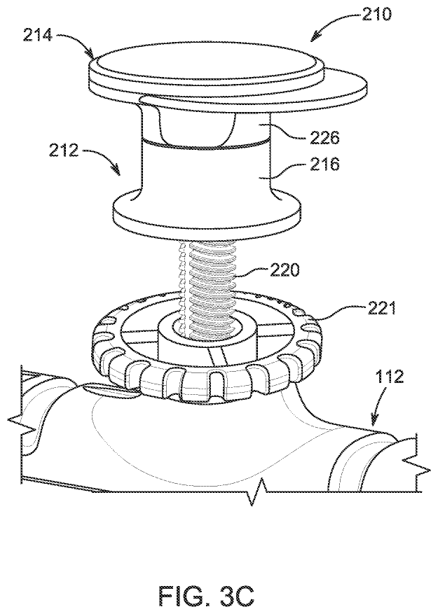

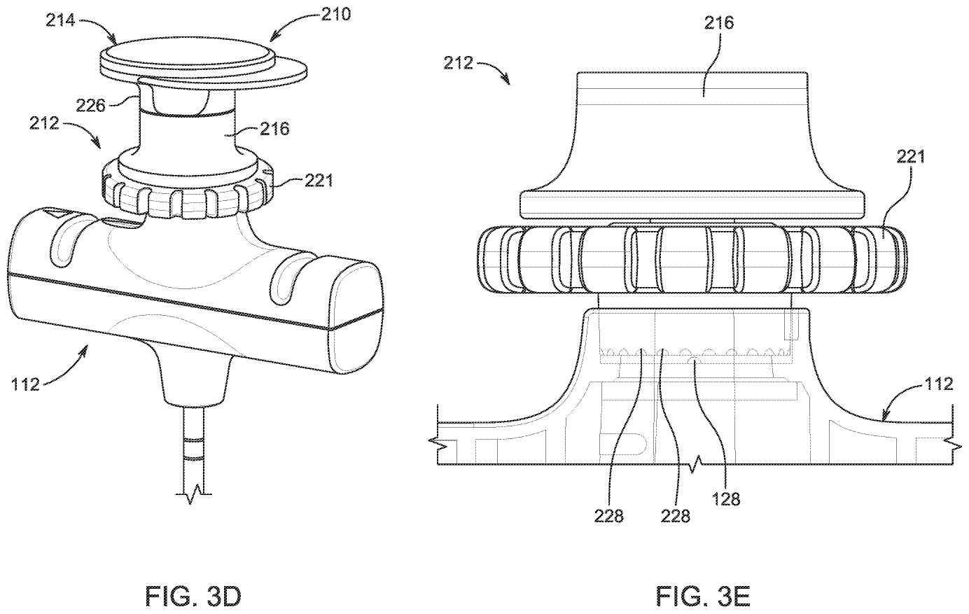

In FIG. 3A, the central lumen of the tube 118 extends from the introducer handle 116 to the open distal tip 122 of the tube 118. The tube 118 may be flared or tapered such that the diameter of the tube 118 is not constant along its entire length. For example, the diameter may decrease abruptly at a certain distance (e.g., 1 cm-3 cm) from a lower edge of the lower portion 113 (e.g., bulged portion or flange) of the introducer handle 116 and then continue with a constant diameter distally of an abrupt flare 119. In another embodiment, the diameter may decrease gradually (e.g., taper uniformly) along the length of the tube 118 from the start of the flare 119 to the open distal tip 122 of the tube 118. The central lumen of the tube 118 may be coated with a medical grade silicone lubricant to improve tool insertion and removal therein. The outer diameter of the tube 118 may range from 3 mm to 5 mm (e.g., from 3.0 mm to 4.0 mm, from 4.0 mm to 4.4 mm, from 4.2 mm to 4.5 mm, from 4.4 mm to 5.0 mm, overlapping ranges thereof, or any value within the recited ranges). The introducer handle 116 may include one or more indicia 130 providing a reference for a direction of curvature of distal end portions of the curved cannula assembly 120 upon insertion within the tube 118 and out of the open distal tip 122 of the tube 118.

As shown in FIG. 3B. The introducer handle 116 may also include one or more first detent or mating elements 128 configured to engage and interact with corresponding second detent or mating features 228 of the curved cannula 212. The one or more first detent or mating features 128 may comprise detents, teeth, nubs, or protrusions extending radially inward from an inner radial surface of a recess in the introducer handle 116 of the introducer cannula 112 against which an outer diameter of a portion of the curved cannula 212 is configured to press or engage upon insertion of the introducer cannula 112 within the upper central opening 120 and the central lumen of the tube 118 of the introducer cannula 112.

FIG. 3A is a side view of the curved cannula 212 of the kit or system 100 of FIG. 1. FIG. 3B is a close-up side view of a proximal end portion of the curved cannula 212.

The curved cannula 212 includes the second handle (e.g., instrument handle) 216, a threaded proximal extension portion 220, a wheel (e.g., gear wheel) 221, and a generally smooth distal portion comprising a rigid support portion 223, and a distal shaft portion 224 which may be more flexible (e.g., made of polymeric material) than the rigid support portion 223. The second handle 216 may include a curved slot and a curved ramp configured to facilitate insertion of the curved stylet 214 into and along a central lumen of the curved cannula 212 extending from the second handle 216 to the open distal tip 222 of the distal shaft portion 224. The central lumen of the curved cannula 212 may be coated with a medical grade silicone lubricant to improve tool insertion and removal.

In the illustrated example, the gear wheel 221 comprises a hole formed coaxial with a central axis of the gear wheel 221. The hole has internal threads formed therein configured to interface with corresponding external threads of the threaded proximal extension portion 220 such that rotation of the gear wheel 221 causes controlled proximal and distal translation of the gear wheel 221 along the threaded proximal extension portion 220. The threaded proximal extension portion 220 is sized such that when the gear wheel 221 is in its distal-most position (as shown in FIG. 3A), the distal tip 222 of the curved cannula 212 does not extend out of the open distal tip 122 of the introducer cannula 112 when the curved cannula assembly 210 is fully inserted therein. The gear wheel 221 may rotate (e.g., spin freely) about the threaded proximal extension portion 220. The threads may comprise triple threads and the gear wheel 221 may be configured to traverse the entire length of the threaded proximal extension portion 220 with four complete rotations of the gear wheel 221. Other thread counts and numbers of rotations are also possible.

The rigid support portion 223 may comprise a biocompatible metal or other rigid material, such as stainless steel, titanium, platinum and/or the like, so as to provide additional support to the curved cannula 212 during insertion of the curved stylet 214 and when the second handle 216 is malleted. The distal shaft portion 224 may be comprised of a thermoplastic, shape-memory polymer material (such as polyether ether ketone (PEEK), polyurethane, polyamide (PA), polycarbonate (PC), polyethylene terephthalate (PET), and/or the like) and the distal end portion 225 is pre-curved (e.g., shape-set) to have a predetermined curvature in a “resting” unconstrained configuration.

Turning to FIG. 3B, the gear wheel 221 includes a lower flange 229 (e.g., annular flange) extending downward from a lower surface of the gear wheel 221. The lower flange 229 can comprise a hole formed in the center with an inner circumferential or annular surface. The inner circumferential surface of the central hole of the lower flange 229 may comprise threading to be engaged with threads on the proximal extension portion 220. The lower flange 229 includes second detent or mating elements 228 corresponding to the one or more first detent or mating elements 128 of the introducer cannula 112. The second detent or mating elements 228 may comprise detent features or elements such as indentations, recesses, notches or formed patterns adapted (e.g., sized and shaped) to mechanically interact or engage with the one or more corresponding mating features or first detent features 128 (e.g., detents) of the introducer cannula 112.

FIGS. 3C and 3D illustrate operation of the gear wheel 221 of the curved cannula 212 to facilitate prevention or advancement of the curved cannula assembly 210 within the introducer cannula 112 at different stages of a bone access method utilizing the kit or system 100 of FIG. 1. As shown in FIG. 3C, the gear wheel 221 is rotated until it is in its distal-most position along the threaded proximal extension portion 220. This gear wheel 221 position may be configured prior to insertion of the curved cannula assembly 210 within the introducer cannula 112 so as to prevent inadvertent advancement of the curved distal end portions 225, 227 of the curved cannula assembly 210 out of the introducer cannula 112. As shown in FIG. 3D, the gear wheel 221 is rotated to its proximal-most position along the threaded proximal extension portion 220 to enable full insertion of the curved cannula assembly 210 within the introducer cannula 112 such that the curved distal end portions 225, 227 of the curved cannula assembly 210 extend out of the introducer cannula 112 and along a curved path within the cancellous bone region of the vertebral body or other bone.

FIG. 3E illustrates a side view of the curved cannula 212 inserted within the introducer cannula 112, with the introducer handle 116 of the introducer cannula 112 being transparent so as to facilitate visualization within. As the lower flange 229 of the gear wheel 221 of the curved cannula 212 is advanced distally within the opening 120 of the introducer cannula 112 by rotation of the gear wheel 221 to move the gear wheel 221 proximally, the second detent elements 228 of the lower flange 229 of the gear wheel 221 eventually can engage or interact with the corresponding one or more first detent 128 of the introducer cannula 112. Once the curved cannula 212 is fully inserted into the introducer cannula 112 and the open distal tip 222 of the curved cannula 212 meets the cancellous tissue in bone, the gear wheel 221 may be disposed a distance above the upper surface of the introducer handle 116 of the introducer cannula 112. When spinning down the gear wheel 221 on the curved cannula 212, the gear wheel 221 spins freely until it comes in contact with the upper face of the introducer cannula 112. Once in range, the second detent elements, e.g., notches, 228 on the gear wheel 221 engage with the one or more first detent features, e.g., teeth, 128 on the top side of the introducer cannula 112. When this engagement occurs, a tactile click can be felt. This tactile click may advantageously allow an operator (e.g., surgeon or other clinician) to identify that the gear wheel 221 is now engaged with the introducer handle 116 of the introducer cannula 112. Once the first click is felt, further rotation of the gear wheel 221 may result in retraction of the curved cannula 212 with respect to open distal tip 122 of the introducer cannula 112. While this retraction is occurring, the tactile click may continue to be felt at regular intervals. If the operator decides to spin or rotate the gear wheel 221 up (e.g., proximally) so as to further advance the curved cannula 212 distally within the introducer cannula 112, once the gear wheel 221 (e.g., second detents 228 of the lower flange 229 of the gear wheel 221) passes out of range of engagement with the first detents 128, the gear wheel 221 may be able to freely spin or rotate again

In accordance with several embodiments, the detent features 128, 228 may advantageously provide for easy tool exchange without loss of axial or longitudinal position. For example, at several points throughout a bone access and/or treatment procedure, introduction and removal of parts (e.g., access instruments, including the curved cannula 212 and curved stylet 214) from the curved cannula 212 may cause inadvertent axial movement of the curved cannula 212 (e.g., further driving the curved cannula 212 into the patient or retracting from the patient.) The detent features 128, 228 may help to prevent this inadvertent, or accidental, movement and may help to maintain part position during tool exchange.

As described previously, the detent features 128, 228 may provide the operator with tactile (and potentially audible) feedback identifying when the gear wheel 221 comes into contact with the upper face of the introducer cannula 112. In addition, when engaged the first detent features 128 may add significant friction to the assembly to prevent accidental movement when exchanging parts but not during normal rotation of the gear wheel 221. This friction can be created in a number of ways including using different shapes (sinusoidal, round, saw-toothed, square, etc.) such as shown in FIGURES SA-5D. The detent features 128, 228 may also act as an anti-rotation/backdrive feature that prevents inadvertent unwinding of the gear wheel 221 when undesired (e.g., during adjustments and/or when applying axial pressure onto the curved cannula 212 when inserting a mating tool such as a treatment probe or a straight stylet. In some implementations, the detent features 128, 228 also may provide the operator more discrete control of the access tools during insertion or retraction.

For example, the operator may have fine control of the rotation of the gear wheel 221, as it can be moved click to click if desired. Without the detent elements 128, 228, the operator would have to visually track the translation of the curved cannula 212 with respect to the introducer cannula 112 by keeping track of how many threads are exposed or by tracking the rotations of the gear wheel 221 (e.g., turning of the gear wheel 221 one rotation or 360 degrees could correspond to 6 mm of axial translation (advancement or retraction)). The detent elements 128, 228 may advantageously allow for a tactile feedback to occur at a fixed rotation (e.g., a tactile bump at every 90 degrees could correspond to a known axial translation (e.g., retraction or advancement) distance). The tactile and/or audible feedback could result in easier communication to the operator or enhanced confidence by the operator that the procedure is being performed properly based on instructions. For example, instead of an instruction that requires visual observation of one or more tool components by the operator, such as “rotate the gear wheel down one and a half turns”, the instruction may be based on tactile and/or audible feedback that does not require visual observation of tool components by the operator (e.g., “turn the gear wheel 6 clicks”).

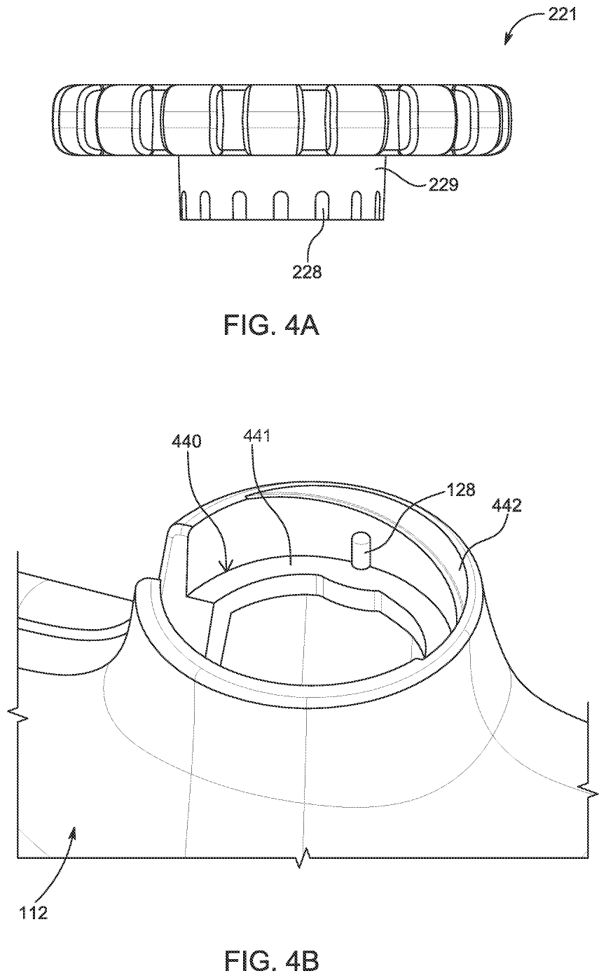

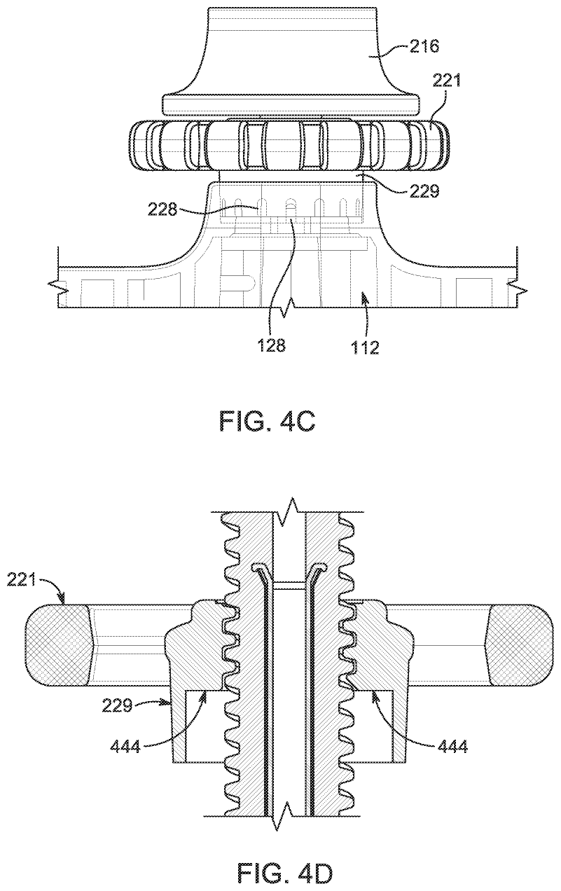

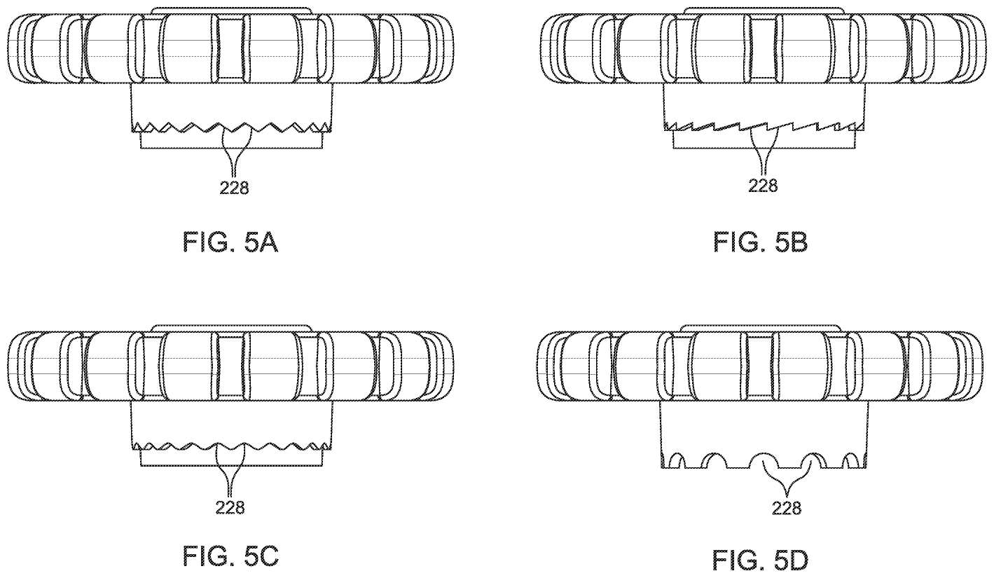

FIGS. 4A and 4B show close-up views of the gear wheel 221 and the upper surface of the introducer cannula 112. FIG. 4A shows a close-up side view of an example of the gear wheel 221 of the curved cannula 212 that includes the lower flange 229 and second detent elements 228. In FIG. 4A, the second detent members 228 are indentations or notches formed around the lateral circumferential surface of the lower flange 229. The number of the second detent elements 228 may vary. The second detent elements 228 may be uniformly spaced, forming a ring of equally spaced detent elements. The number of the detent elements 228 and spacing of the second detent elements 228 may be adapted to correspond to a certain axial translation distance of the gear wheel 221 along the threaded extension portion 220 of the curved cannula 212 or to correspond to a certain degree amount of rotation of the gear wheel 221. For example, the operator may be instructed that each click of rotation of the gear wheel 221 is equivalent to a certain angle and a certain translation distance of the gear wheel 221 along the threaded extension portion 220. The detent elements may not be equally spaced in other examples.

FIG. 4B shows a perspective top view of a portion of the introducer handle 116 of the introducer cannula 112 illustrating an example of a first detent element 128 of the introducer cannula 112. The opening 120 of the introducer cannula 112 may include an abutment member 440 extending inwardly from an inner diameter of the opening 120 so as to provide an upper surface with which a lower surface of the lower flange 229 of the gear wheel 221 may contact. The abutment member 440 may be one of the coupling features 121 of the introducer cannula 112. The abutment member 440 may comprise a ledge that extends continuously around at least a portion of a circumference of the opening 120 of the introducer cannula 112. The abutment member 440 may extend around an entire circumference of the opening 120 except for the portion defined by a lateral slot adapted to facilitate insertion of the curved distal end portions of the curved cannula assembly 210. The abutment member 440 may alternatively not be continuous and may comprise discrete, separate members positioned around the circumference. The abutment member 440 may provide friction as the gear wheel 221 is rotated. As shown in FIG. 4B, the first detent element 128 may extend upward from the abutment member 440. The first detent element 128 may be a tooth or protrusion located at or close to a lateral circumferential surface of the opening 120, configured to bite in or mate with one of the indentations of the second detent elements 228.

The opening 120 may also include an internal ramp 442 configured to provide a mechanical advantage to assist in removal (e.g., reduction in removal force) of the introducer stylet 114 from the introducer cannula 112 (especially if bone shards have packed into gaps between the introducer stylet 114 and introducer cannula 112 making removal more difficult) as the first handle 126 is rotated (e.g., 120-degree rotation counter-clockwise). The introducer cannula 112 may include a single first detent element (e.g., a tooth) 128 or may include multiple first detent elements 128. The number and position of the detent elements 128 may vary. As one example, there may be two first detent elements 128 positioned 180 degrees or substantially 180 degrees apart. The first detent elements 128 (e.g. teeth, notches, grooves, tabs) may additionally or alternatively appear on a proximal (or upwardly-facing) abutment surface 441 formed by the abutment member 440 and interact with a bottom surface (e.g., corresponding second detent elements 228 on the bottom surface) of the lower flange 229 of the gear wheel 221. FIG. 4D shows a side cross-section view of a proximal portion of an implementation of the curved cannula 212 including the gear wheel 221. With reference to FIG. 2B and FIG. 4D, in some implementations, a proximal or upwardly-facing face of the opening 120 of the tube 118 of the introducer cannula 112 may have first detent elements 128 (not shown) formed to interact with an inner plane 444 of the gear wheel 221 (e.g., corresponding second detent elements 228 on an inner plane of the lower flange 229 of the gear wheel 221).

FIG. 4C illustrates a side view of the curved cannula 212 including the gear wheel 221 shown in FIG. 4A inserted within the introducer handle 116 of the introducer cannula 112 shown in FIG. 4B, with the introducer cannula 112 being transparent. As the gear wheel 221 is rotated to cause the gear wheel 221 to translate distally, the one or more first detent elements 128 of the introducer cannula 112 will eventually engage with the second detent elements 228 of the lower flange 229 of the gear wheel 221. FIG. 4C shows a first detent element 128 of the introducer cannula 112 is engaged with a second detent element 228 of the lower flange 229 of the gear wheel 221.

The shape and arrangement of the first detent element(s) 128 on the introducer handle 116 of the introducer cannula 112 and the shape and arrangement of the second detent elements 228 on the lower flange 229 of the curved cannula 114 shown in FIGS. 4A-4D can be reversed to achieve the same detent function. For example, the first detent elements 128 can be a ring of equally spaced indentations formed on the abutment surface 441 of the abutment 440 or on the lateral circumferential surface of the opening 128. The second detent elements 228 can be a tooth or two or more teeth formed on the distal surface of the lower flange 229 or on the lateral circumferential surface to bite in or mate with the corresponding indentations of the first detent elements 128.

FIGURES SA, 5B, 5C, and 5D illustrate additional examples of the detent elements 228 of the gear wheel 221 of the curved cannula 212. FIGURE SA shows second detent elements 228 having a triangular shape; FIG. 5B shows second detent elements 228 having a sawtooth shape; FIG. 5C shows second detent elements 228 having a sinusoidal shape; and FIG. 5D shows second detent elements 228 having a semi-circular shape. Other different shapes may be used as desired and/or required. The different shapes may provide different amounts of friction and different levels of tactile or audible feedback. In accordance with several embodiments, each of the second detent elements 228 in FIGS. 5A-5D takes a ring shape with equally spaced detent elements around the circumference of the lower flange 229 of the curved cannula 114. In some implementations, the detent elements in ring shape can be disposed in the opening 120 of the introducer handle 116 of the introducer cannula 112.

In accordance with several embodiments, the detent or mating elements 128, 228 may comprise structures other than formed features in mating plastic components (e.g., detents or detent elements or features). The other mating structures may still provide one or more of the benefits of the detent elements or features described above. The other mating structures may not include a physical engagement or mating.

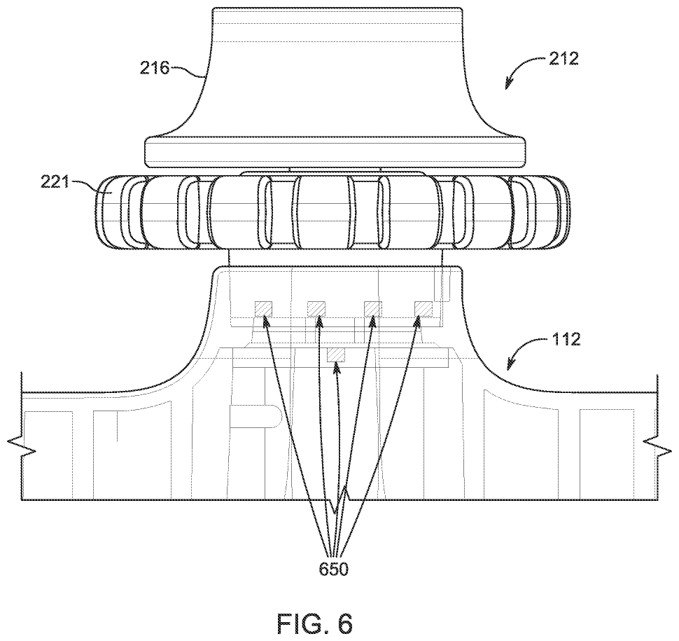

With reference to FIG. 6, the other mating structures could include magnets 650 of the same or opposite polarity adapted to create tactile feedback (change in feeling or resistance) when in range (e.g., close enough proximity to magnetically interact). The mating structure may also add additional force needed to avoid accidentally moving (e.g., advance or retract) the curved cannula 212 with respect to the introducer cannula 112. The number of magnets 650 may vary as desired and/or required.

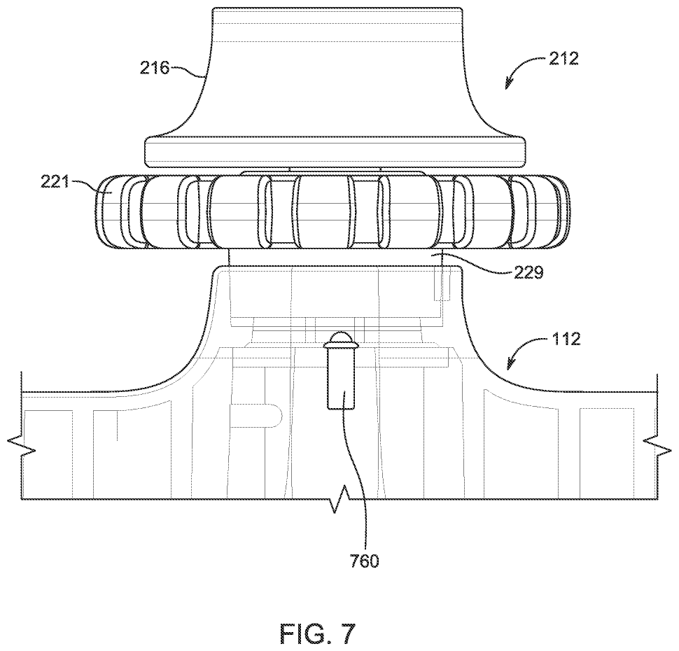

In various implementations, other mating structures may incorporate use of one or more leaf springs, wave springs, or coil springs and/or molded plastic to cause a ball or lever arm to create a tactile effect and/or audible click or sound capable of being perceived by an operator. FIG. 7 schematically illustrates a spring detent mechanism 760 adapted to cause a ball on a lever arm to engage with corresponding mating features (e.g., second detents 228 not shown) on the lower flange 229 of the gear wheel 221 of the curved cannula 212. Alternatively, a leaf spring, wave spring or compression spring could be used to apply force to a detent diametrically around the gear wheel 221. An axial or radial friction clutch mechanism may alternatively be implemented. Active mechanisms could also be employed, such as a button lock, bayonet lock, pull pin, etc. to prevent movement between the various tools upon insertion and retraction and to provide a change of tactile feeling upon interaction of components of the curved cannula 212 and the introducer cannula 112.

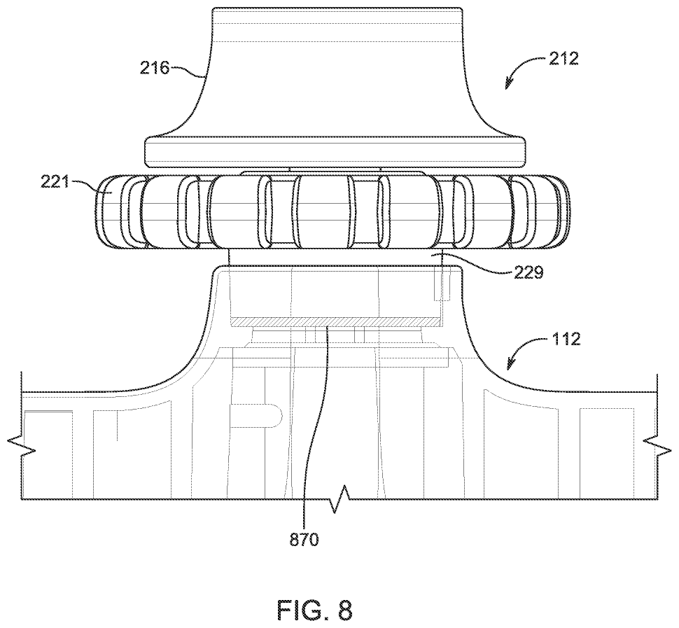

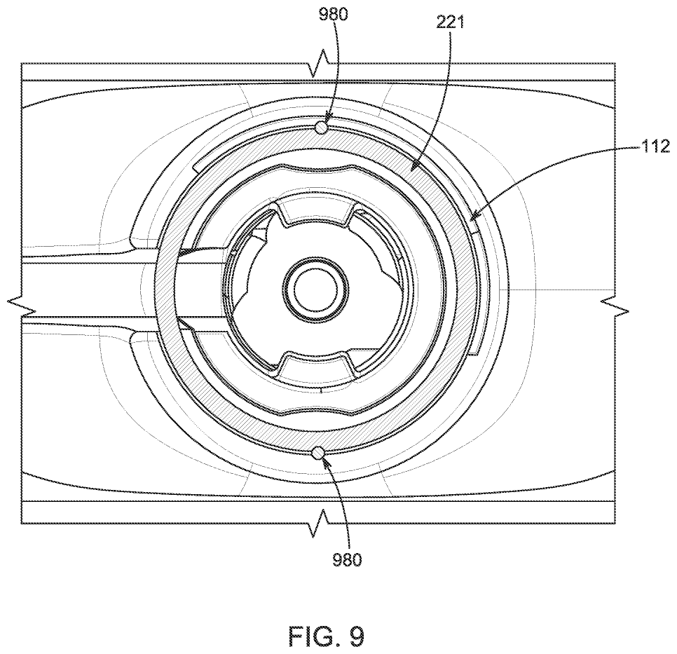

In some implementations, an interfacing material may be incorporated to change the tactile feedback as the gear wheel 221 comes in contact with the introducer cannula 112. This could present in a change of input force needed by the operator (e.g. until the gear wheel 221 comes in contact there is minimal torque required to advance, however, once in range additional or increasing torque could be required to advance.) Examples of such an interfacing mechanism could involve the use of an elastomeric material, hook and loop fasteners, and/or interference fits between the parts or components. FIG. 8 shows an embodiment incorporating an elastomeric ring 870 positioned on a lower contact surface of or within an inner diameter of the lower flange 229 of the gear wheel 221 to provide an interface with features of the introducer cannula 112. FIG. 9 schematically illustrates a top cross-sectional view of the introducer cannula 112 with the gear wheel 221 of the curved cannula 212 engaged with the introducer cannula 112. The interference mechanism (e.g., one or more first mating features 980) may include one or more interference features to provide a friction or interference fit between the components. The one or more interference features can be made from the same material as the gear wheel 221 or an elastomer or other material.

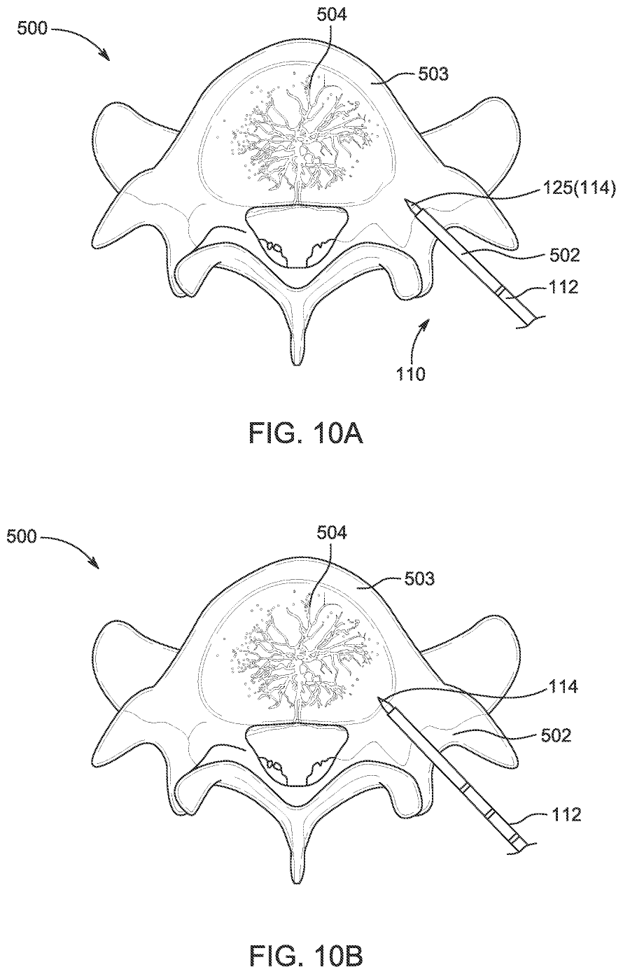

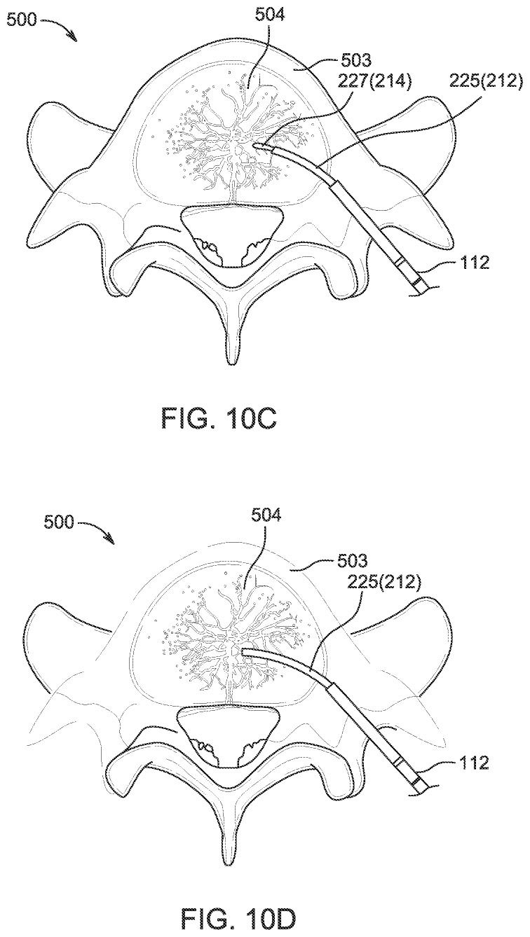

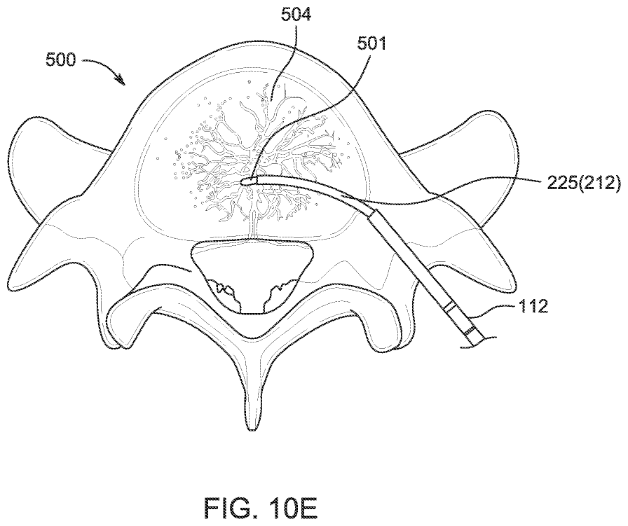

FIGS. 10A-10E illustrate an embodiment of steps of a method of using the access tools to facilitate percutaneous access (e.g., minimally invasive through a percutaneous incision) to a location within a vertebral body 500 for treatment (e.g., modulation of intraosseous nerves, such as a basivertebral nerve, bone cement delivery for treatment of vertebral fractures, and/or ablation of bone tumors). With reference to FIG. 10A, the distal portion of the introducer assembly 110 (including the distal tip 125 of the introducer stylet 114 and the distal tip 122 of the introducer cannula 112) are inserted (via a percutaneous incision in the skin) through a pedicle 502 adjacent the vertebral body 500 by malleting on the first handle 126 of the introducer stylet 114 after insertion and aligned engagement of the introducer stylet 114 within the introducer cannula 112.

With reference to FIG. 10B, the introducer assembly 110 may then be malleted so as to advance the distal tip 122 of the introducer cannula 112 to the entry site into (or within) the cancellous bone region 504 of the vertebral body 500. The introducer stylet 114 may then be removed from the introducer cannula 112.

The curved cannula assembly 210 may then be inserted within the introducer cannula 112 with the gear wheel 221 in the distal-most position so as to prevent inadvertent advancement of the curved cannula assembly 210 out of the open distal tip 122 of the introducer cannula 112 prematurely. With reference to FIG. 10C, after rotation of the gear wheel 221 to transitionally or translationally move the gear wheel 221 to a more proximal position, the curved cannula assembly 210 can be malleted so as to advance the collective curved distal end portions 225, 227 of the curved cannula assembly 210 together out of the distal tip 122 of the introducer cannula 112 and along a curved path within the cancellous bone region 504. As described above, upon advancement of the curved cannula assembly 210 within the introducer cannula 112 the detent elements 128, 228 or other mating structures will eventually engage each other. Subsequently, the detent elements 128, 228 may facilitate controlled movement and provide tactile and/or audible feedback. With reference to FIG. 10D, the curved stylet 214 may then be removed from the curved cannula 212, with the curved cannula 212 remaining in position. In accordance with several embodiments, the path formed by the prior instruments may advantageously allow the curved cannula assembly 210 to have a head start and begin curving immediately upon exiting the open distal tip 122 of the introducer cannula 112.

If a further straight path beyond the curved path is desired to reach a target treatment location, a straight stylet may be inserted through the curved cannula 212 such that the distal channeling tip of the straight stylet extends beyond the open distal tip of the curved cannula 212 and along a straight path toward the target treatment location (e.g., a basivertebral nerve trunk or basivertebral foramen). In some embodiments, the straight stylet may not be needed and this step may be skipped.

With reference to FIG. 10E, a treatment device 501 (e.g., a flexible bipolar radiofrequency probe) may be inserted through the curved cannula 212 (after removal of the straight stylet if used or the curved stylet 214) and advanced out of the open distal tip of the curved cannula 212 to the target treatment location. The treatment device 501 may then perform the desired treatment. For example, if the treatment device 501 is a radiofrequency probe, the treatment device 501 may be activated to modulate (e.g., ablate, denervate, stimulate) intraosseous nerves (e.g., a basivertebral nerve or other intraosseous nerve within a vertebral body or innervating a vertebral endplate) or a tumor within the vertebral body 500. Bone cement or other agent, or a diagnostic device (such as a nerve stimulation device or an imaging device to confirm ablation of a nerve) may optionally be delivered through the curved cannula 212 after the treatment device 501 is removed from the curved cannula 212. As discussed above, the detent or other mating features 128, 228 may prevent inadvertent movement of the curved cannula 212 with respect to the introducer cannula 112 during insertion and removal of the curved stylet 214, straight stylet or treatment device 501 into and from the curved cannula 212.

The treatment devices (e.g., treatment probes) may be any device capable of modulating tissue (e.g., nerves, tumors, bone tissue). Any energy delivery device capable of delivering energy can be used (e.g., RF energy delivery devices, microwave energy delivery devices, laser devices, infrared energy devices, other electromagnetic energy delivery devices, ultrasound energy delivery devices, and the like). The treatment device 501 may be an RF energy delivery device. The RF energy delivery device may include a bipolar pair of electrodes at a distal end portion of the device. The bipolar pair of electrodes may include an active tip electrode and a return ring electrode spaced apart from the active tip electrode. The RF energy delivery device may include one or more temperature sensors (e.g., thermocouples, thermistors) positioned on an external surface of, or embedded within, a shaft of the energy delivery device. The RF energy delivery device may not employ internally circulating cooling, in accordance with several implementations

In some implementations, water jet cutting devices may be used to modulate (e.g., denervate) nerves. For example, a water jet cutter may be configured to generate a very fine cutting stream formed by a very high-pressure jet of water. For example, the pressure may be in the range of 15 MPa to 500 MPa (e.g., 15 MPa to 50 MPa, 30 MPa-60 MPa, 50 MPa-100 MPa, 60 MPa-120 MPa, 100 MPa-200 MPa, 150 MPa-300 MPa, 300 MPa-500 MPa, overlapping ranges thereof, or any value within the recited ranges). In some implementations, a chemical neuromodulation tool injected into a vertebral body or at an endplate may be used to ablate or otherwise modulate nerves or other tissue. For example, the chemical neuromodulation tool may be configured to selectively bind to a nerve or endplate. In some implementations, a local anesthetic (e.g., liposomal local anesthetic) may be used inside or outside a vertebral body or other bone to denervate or block nerves. In some implementations, brachytherapy may be used to place radioactive material or implants within the vertebral body to deliver radiation therapy sufficient to ablate or otherwise denervate the vertebral body. In some implementations, chymopapain injections and/or condoliase injections may be used (e.g., under local anesthesia). Phototherapy may be used to ablate or otherwise modulate nerves after a chemical or targeting agent is bound to specific nerves or to a vertebral endplate.

In accordance with several implementations, thermal energy may be applied within a cancellous bone portion (e.g., by one or more radiofrequency (RF) energy delivery instruments coupled to one or more RF generators) of a vertebral body. The thermal energy may be conducted by heat transfer to the surrounding cancellous bone, thereby heating up the cancellous bone portion. In accordance with several implementations, the thermal energy is applied within a specific frequency range and having a sufficient temperature and over a sufficient duration of time to heat the cancellous bone such that the basivertebral nerve extending through the cancellous bone of the vertebral body is modulated. In several implementations, modulation comprises permanent ablation or denervation or cellular poration (e.g., electroporation). In some implementations, modulation comprises temporary denervation or inhibition. In some implementations, modulation comprises stimulation or denervation without necrosis of tissue.

For thermal energy, temperatures of the thermal energy may range from about 70 to about 115 degrees Celsius (e.g., from about 70 to about 90 degrees Celsius, from about 75 to about 90 degrees Celsius, from about 83 to about 87 degrees Celsius, from about 80 to about 100 degrees Celsius, from about 85 to about 95 degrees Celsius, from about 90 to about 110 degrees Celsius, from about 95 to about 115 degrees Celsius, or overlapping ranges thereof). The temperature ramp may range from 0.1-5 degrees Celsius/second (e.g., 0.1-1.0 degrees Celsius/second, 0.25 to 2.5 degrees Celsius/second, 0.5-2.0 degrees Celsius/second, 1.0-3.0 degrees Celsius/second, 1.5-4.0 degree Celsius/second, 2.0-5.0 degrees Celsius/second). The time of treatment may range from about 10 seconds to about 1 hour (e.g., from 10 seconds to 1 minute, 1 minute to 5 minutes, from 5 minutes to 10 minutes, from 5 minutes to 20 minutes, from 8 minutes to 15 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy may be delivered as an alternative to or in sequence with continuous energy. For radiofrequency energy, the energy applied may range from 350 kHz to 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 kHz±5 kHz, 475 kHz±5 kHz, 487 kHz #5 kHz). A power of the radiofrequency energy may range from 5 W to 30 W (e.g., from 5 W to 15 W, from 5 W to 20 W, from 8 W to 12 W, from 10 W to 25 W, from 15 W to 25 W, from 20 W to 30 W, from 8 W to 24 W, and overlapping ranges thereof, or any value within the recited ranges). In accordance with several implementations, a thermal treatment dose (e.g., using a cumulative equivalent minutes (CEM) 43 degrees Celsius thermal dose calculation metric model) is between 200 and 300 CEM (e.g., between 200 and 240 CEM, between 230 CEM and 260 CEM, between 240 CEM and 280 CEM, between 235 CEM and 245 CEM, between 260 CEM and 300 CEM) or greater than a predetermined threshold (e.g., greater than 240 CEM). The CEM number may represent an average thermal cumulative dose value at a target treatment region or location and may represent a number that expresses a desired dose for a specific biological end point. Thermal damage may occur through necrosis or apoptosis.

Cooling may optionally be provided to prevent surrounding tissues from being heated during the nerve modulation procedure. The cooling fluid may be internally circulated through the delivery device from and to a fluid reservoir in a closed circuit manner (e.g., using an inflow lumen and an outflow lumen). The cooling fluid may comprise pure water or a saline solution having a temperature sufficient to cool electrodes (e.g., 2-10 degrees Celsius, 5-10 degrees Celsius, 5-15 degrees Celsius). Cooling may be provided by the same instrument used to deliver thermal energy (e.g., heat) or a separate instrument. In accordance with several implementations, cooling is not used.

In some implementations, ablative cooling may be applied to the nerves or bone tissue instead of heat (e.g., for cryoneurolysis or cryoablation applications). The temperature and duration of the cooling may be sufficient to modulate intraosseous nerves (e.g., ablation, or localized freezing, due to excessive cooling). The cold temperatures may destroy the myelin coating or sheath surrounding the nerves. The cold temperatures may also advantageously reduce the sensation of pain. The cooling may be delivered using a hollow needle under fluoroscopy or other imaging modality.

In some implementations, one or more fluids or agents may be delivered to a target treatment site to modulate a nerve. The agents may comprise bone morphogenetic proteins, for example. In some implementations, the fluids or agents may comprise chemicals for modulating nerves (e.g., chemoablative agents, alcohols, phenols, nerve-inhibiting agents, or nerve stimulating agents). The fluids or agents may be delivered using a hollow needle or injection device under fluoroscopy or other imaging modality.

One or more treatment devices (e.g., probes) may be used simultaneously or sequentially. For example, the distal end portions of two treatment devices may be inserted to different locations within a vertebral body or other bone or within different vertebral bodies or bones. Radiofrequency treatment probes may include multiple electrodes configured to act as monopolar, or unipolar, electrodes or as pairs of bipolar electrodes. The treatment device(s) may also be pre-curved or curveable such that the curved stylet is not needed or may have sharp distal tips such that additional sharpened stylets are not needed. In some implementations, any or all of the access tools and the treatment devices are MR-compatible so as to be visualized under MR imaging.

In accordance with several embodiments, a method of facilitating ablation of one or more nerves within a vertebral body includes applying radiofrequency energy to a location within the vertebral body according to the following treatment parameters: a frequency between 400 kHz and 600 kHz (e.g., between 400 kHz and 500 kHz, between 450 kHz and 500 kHz, between 470 kHz and 490 kHz, between 500 kHz and 600 kHz, overlapping ranges thereof, or any value within the recited ranges); a target temperature of between 60 degrees Celsius and 90 degrees Celsius (e.g., between 60 and 80 degrees Celsius, between 65 and 75 degrees Celsius, between 70 and 80 degrees Celsius, between 80 degrees and 90 degrees Celsius, overlapping ranges thereof, or any value within the recited ranges); a temperature ramp of between 0.5 and 3 degrees Celsius per second (e.g., between 0.5 and 1.5 degrees Celsius per second, between 1.0 and 2.0 degrees Celsius per second, between 1.5 and 3 degrees Celsius per second, overlapping ranges thereof, or any value within the recited ranges, such as 0.5 degree Celsius per second, 1 degree Celsius per second, 1.5 degrees Celsius per second, 2 degrees Celsius per second, 2.5 degrees Celsius per second, 3 degrees Celsius per second), and an active energy delivery time of between 1 minute and 20 minutes (e.g., between 1 minute and 5 minutes, between 5 minutes and 15 minutes, between 10 minutes and 20 minutes, between 10 and 15 minutes, overlapping ranges thereof, or any value within the recited ranges).

In some implementations, the gear wheel may be substituted with another translation mechanism, such as a sliding mechanism, a ratchet mechanism, a squeeze-and-pull mechanism, and/or the like.

### CONCLUSION

In some implementations, the system comprises various features that are present as single features (as opposed to multiple features). For example, in one embodiment, the system includes a single radiofrequency generator, a single introducer cannula with a single stylet, a single radiofrequency energy delivery device or probe, and a single bipolar pair of electrodes. A single thermocouple (or other means for measuring temperature) may also be included. Multiple features or components are provided in alternate embodiments.

In some implementations, the system comprises one or more of the following: means for tissue modulation (e.g., an ablation or other type of modulation catheter or delivery device), means for monitoring temperature (e.g., thermocouple, thermistor, infrared sensor), means for imaging (e.g., MRI, CT, fluoroscopy), means for accessing (e.g., introducer assembly, curved cannulas, drills, curettes), means for actuating (e.g., threaded knob or screw actuation mechanism, sliding actuator, pullwire actuator, lever, hydraulic actuator, pneumatic actuator, electrical actuator, push button actuator, mechanical linear actuator, etc.)

Although certain embodiments and examples have been described herein, aspects of the methods and devices shown and described in the present disclosure may be differently combined and/or modified to form still further embodiments. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. The section headings used herein are merely provided to enhance readability and are not intended to limit the scope of the embodiments disclosed in a particular section to the features or elements disclosed in that section.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “applying thermal energy” include “instructing the applying of thermal energy.”

The terms “top,” “bottom,” “first,” “second,” “upper,” “lower,” “height,” “width,” “length,” “end,” “side,” “horizontal,” “vertical,” and similar terms may be used herein; it should be understood that these terms have reference only to the structures shown in the figures and are utilized only to facilitate describing embodiments of the disclosure. The terms “proximal” and “distal” are opposite directional terms. For example, the distal end of a device or component is the end of the component that is furthest from the operator during ordinary use. A distal end or tip does not necessarily mean an extreme distal terminus. The proximal end refers to the opposite end, or the end nearest the operator during ordinary use. Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges such as from 70 to 80 degrees, from 70 to 100 degrees, from 70 to 110 degrees, from 80 to 100 degrees etc., as well as individual numbers within that range, for example, 70, 80, 90, 95, 100, 70.5, 90.5 and any whole and partial increments therebetween. Language such as “up to,” “at least,” “greater than,” “less than,” “between,” and the like includes the number recited. Numbers preceded by a term such as “about” or “approximately” include the recited numbers. For example, “about 2:1” includes “2:1.” For example, the terms “approximately”, “about”, and “substantially” as used herein represent an amount close to the stated amount that still performs a desired function or achieves a desired result.

What is claimed is:

1-79. (canceled)

80. A bone access system adapted to facilitate percutaneous access to a target treatment location within bone, the system comprising:
an introducer cannula comprising a proximal handle and a distal elongate tube extending from the proximal handle;
wherein the proximal handle of the introducer cannula comprises a central opening in its upper surface,
wherein the central opening of the proximal handle of the introducer cannula comprises one or more detents projecting radially inward from an inner surface of the central opening, and
wherein the distal elongate tube of the introducer cannula comprises a lumen accessible via the central opening of the proximal handle of the introducer cannula; and
an access instrument sized and configured to be inserted through the central opening and along the lumen of the introducer cannula until a distal end portion of the access instrument extends beyond an open distal tip of the introducer cannula,
wherein the access instrument comprises an elongate shaft having a threaded proximal portion and a generally smooth distal portion and a gear wheel mechanically coupled to the threaded proximal portion and adapted to be translated proximally and distally along the threaded proximal portion via rotation of the gear wheel,
wherein the gear wheel comprises an annular flange, and
wherein the annular flange comprises a plurality of detent elements spaced around a circumference of a lower surface of the annular flange adapted to mechanically engage with the one or more detents within the central opening of the proximal handle of the introducer cannula so as to provide tactile and/or audible feedback to an operator upon engagement of the plurality of detent elements of the access instrument with the one or more detents of the introducer cannula so as to provide controlled advancement of the access instrument with respect to the introducer cannula.

81. The bone access system of claim 80, wherein the plurality of detent elements of the annular flange comprises notches or recesses.

82. The bone access system of claim 80, wherein the one or more detents comprises protrusions formed along the inner surface of the central opening.

83. The bone access system of claim 80, wherein the one or more detents and the plurality of detent elements produce an audible click upon engagement and/or disengagement.

84. The bone access system of claim 80, wherein engagement between the one or more detents and a respective one of the plurality of detent elements results in an increase in an amount of torque required to disengage the one or more detents from the respective one of the plurality of detent elements.

85. The bone access system of claim 80, wherein the plurality of detent elements are uniformly spaced around a circumference of the annular flange of the gear wheel of the access instrument.

86. The bone access system of claim 80, wherein the proximal handle of the introducer cannula comprises a curved insertion slot.

87. An introducer cannula for penetrating into a bone, comprising:
an introducer handle at a proximal end, the introducer handle comprising an abutment surface disposed about an upper surface, the abutment surface having a first detent feature formed thereon;
a distal elongated tube attached to and extending from the introducer handle along a longitudinal axis;
wherein a lumen extends through the introducer cannula from the introducer handle to an open distal tip of the distal elongated tube along the longitudinal axis; and
wherein the first detent feature is configured to mate with a second detent feature formed on an access instrument.

88. The introducer cannula of claim 8Error! Reference source not found., wherein when the first detent feature on the abutment surface is configured to mate with the second detent feature on the access instrument, rotation of the second detent feature around the longitudinal axis against the first detent feature causes a tactile response.

89. The introducer cannula of claim 87Error! Reference source not found., wherein when the first detent feature on the abutment surface is configured to mate with the second detent feature on the access instrument, rotation of the second detent feature around the longitudinal axis against the first detent feature results in an audible clicking response.

90. The introducer cannula of claim 87Error! Reference source not found., wherein when the first detent feature on the abutment surface is configured to mate with the second detent feature on the access instrument, rotation of the second detent feature around the longitudinal axis against the first detent feature needs to overcome a torque of a predetermined amount to cause disengagement of the second detent feature and the first detent feature.

91. The introducer cannula of claim 87Error! Reference source not found., wherein the first detent feature on the abutment surface of the introducer handle is one or more teeth and the second detent feature on the access instrument is a plurality of indentations, wherein when the first detent feature is mated with the second detent feature each of the one or more teeth bites into one of the plurality of the indentations.

92. The introducer cannula of claim 87Error! Reference source not found., wherein the first detent feature on the abutment surface of the introducer handle is a plurality of indentations and the second detent feature on the access instrument is one or more teeth, wherein when the first detent feature is mated with the second detent feature each of the one or more teeth bites into one of the plurality of the indentations.

93. The introducer cannula of claim 87Error! Reference source not found., wherein the plurality of indentations forms a circle around the longitudinal axis.

94. The introducer cannula of claim 87Error! Reference source not found., wherein the plurality of indentations are formed on a cylindrical lateral circumferential surface coaxial with the longitudinal axis.

95. A method for facilitating percutaneous access to a target treatment location within a bone, the method comprising:
inserting a distal portion of an introducer assembly to at least an outer cortical region of the bone, wherein the introducer assembly comprises an introducer cannula and an introducer stylet, the introducer cannula having a proximal introducer handle at a proximal end and a distal elongated tube attached to and extending from the introducer handle, the elongated tube defining a longitudinal axis, the introducer cannula having a lumen formed therethrough from the proximal introducer handle to a distal tip of the elongated tube, the introducer stylet configured to be received into the lumen;
removing the introducer stylet from the lumen of the introducer cannula;
inserting a curved cannula assembly into the lumen, wherein the curved cannula assembly comprises a curved cannula handle and a shaft portion extended from the curved cannula handle, the shaft portion configured to be received into the lumen, the shaft portion having a distal curved portion;
advancing the distal curved portion of the curved cannula assembly out of the distal tip of the introducer cannula toward the target treatment location within the bone; and
wherein the introducer handle has a first detent feature formed thereon, and wherein the curved cannula handle has a second detent feature formed thereon configured to mate with the first detent feature so as to provide tactile and/or audible feedback to an operator upon engagement of the second detent feature and the first detent feature so as to provide controlled advancement of the curved cannula assembly with respect to the introducer cannula.

96. The method of claim 95, wherein the mating between the first detent feature of the introducer handle and second detent feature of the curved cannula handle allows a radial or axial positioning of the curved cannula assembly within the introducer cannula.

97. The method of claim 95, wherein the curved cannula assembly further comprises a gearwheel coupled to the curved cannula handle, wherein a lower flange of the gearwheel comprises the second detent feature, wherein the gearwheel is configured to adjust an axial position on the curved cannula handle, and wherein the engagement of the gearwheel with the introducer handle defines the longitudinal position of the curved cannula assembly within the introducer cannula.

98. The method of claim 18, wherein continued rotation of the gearwheel provides further tactile and/or audible feedback via the interaction of the first detent feature with the second detent feature.

99. The method of claim 95, further comprising advancing a treatment instrument through the access instrument and performing a treatment procedure within the bone, the treatment procedure comprising applying radiofrequency energy sufficient to ablate one or more nerves within the bone.
